(19) United States
(12) Patent Application Publication
(10) Pub. No.: US 2010/0114142 A1
(43) Pub. Date: May 6, 2010

(54) POWERING IMPLANTABLE DISTENSION SYSTEMS USING INTERNAL ENERGY HARVESTING MEANS

(76) Inventors: Thomas E. ALBRECHT, Cincinnati, OH (US); Jason L. HARRIS, Mason, OH (US); Mark S. ORTIZ, Milford, OH (US); Michael J. STOKES, Cincinnati, OH (US); Mark S. ZEINER, Mason, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/261,089

(22) Filed: Oct. 30, 2008

(51) Int. Cl. A61M 29/02

(52) U.S. Cl. 606/191

(57) ABSTRACT

An implant for placement within a hollow body organ. The implant includes a distension device having an undeployed shape for delivery within a hollow body and one or more deployed shapes for implantation therein. The distension device has sufficient rigidity in its deployed shape to exert an outward force against an interior of the hollow body so as to bring together two substantially opposing surfaces of the hollow body. The implant also includes a powered means for changing the deployed shape of the member while implanted within the hollow body including an apparatus operable to convert energy produced by the patient into energy to power the implantable device.

This case is related to the following commonly assigned and concurrently filed U.S. applications, all of which are hereby incorporated herein by reference:

U.S. Ser. No. ______ (Attorney Docket Number END6514USNP) titled DEVICES and METHODS FOR ADJUSTING A SATIATION AND SATIETY-INDUCING IMPLANTED DEVICE; U.S. Ser. No. ______ (Attorney Docket Number END6515USNP) titled Sensor Trigger; U.S. Ser. No. ______ (Attorney Docket Number END6516USNP) titled AUTOMATICALLY ADJUSTING INTRA-GASTRIC SATIATION AND SATIETY CREATION DEVICE; U.S. Ser. No. ______ (Attorney Docket Number END6517USNP) titled OPTIMIZING THE OPERATION OF AN INTRA-GASTRIC SATIETY CREATION DEVICE; U.S. Ser. No. ______ (Attorney Docket Number END6518USNP) titled POWERING IMPLANTABLE DISTENSION SYSTEMS USING INTERNAL ENERGY HARVESTING MEANS; U.S. Ser. No. ______ (Attorney Docket Number END6519USNP) titled WEARABLE ELEMENTS FOR INTRA-GASTRIC SATIETY CREATION SYSTEMS; U.S. Ser. No. ______ (Attorney Docket Number END6520USNP) titled INTRA-GASTRIC SATIETY CREATION DEVICE WITH DATA HANDLING DEVICES AND METHODS; U.S. Ser. No. ______ (Attorney Docket Number END6521USNP) titled GUI FOR AN IMPLANTABLE DISTENSION DEVICE AND A DATA LOGGER; U.S. Ser. No. ______ (Attorney Docket Number END6522USNP) titled METHODS AND DEVICES FOR FIXING ANTENNA ORIENTATION IN AN INTRA-GASTRIC SATIETY CREATION SYSTEM; U.S. Ser. No. ______ (Attorney Docket Number END6523USNP) titled METHODS AND DEVICES FOR PREDICTING INTRA-GASTRIC SATIETY CREATION DEVICE SYSTEM PERFORMANCE; U.S. Ser. No. ______ (Attorney Docket Number END6524USNP) titled CONSTANT FORCE MECHANISMS for Regulating Distension Devices; U.S. Ser. No. ______ (Attorney Docket Number END6525USNP) titled A METHOD OF REMOTELY ADJUSTING A SATIATION AND SATIETY-INDUCING IMPLANTED DEVICE.

### FIELD OF THE INVENTION

The present invention relates to methods and devices for providing power to implantable distension systems.

### BACKGROUND OF THE INVENTION

Obesity is becoming a growing concern, particularly in the United States, as the number of obese people continues to increase, and more is learned about the negative health effects of obesity. Morbid obesity, in which a person is 100 pounds or more over ideal body weight, in particular poses significant risks for severe health problems. Accordingly, a great deal of attention is being focused on treating obese patients One proposed method of treating morbid obesity has been to place a distension device, such as a, spring loaded coil inside the stomach. Examples of satiation and satiety inducing gastric implants, optimal design features, as well as methods for installing and removing them are described in commonly owned and pending U.S. patent application Ser. No. 11/469,564, filed Sep. 1, 2006, and pending U.S. patent application Ser. No. 11/469,562, filed Sep. 1, 2006, which are hereby incorporated herein by reference in their entirety. One effect of the coil is to more rapidly induce feelings of satiation defined herein as achieving a level of fullness during a meal that helps regulate the amount of food consumed. Another effect of the coil is to prolong the effect of satiety which is defined herein as delaying the onset of hunger after a meal which in turn regulates the frequency of eating. By way of a non-limiting list of examples, positive impacts on satiation and satiety may be achieved by an intragastric coil through one or more of the following mechanisms: reduction of stomach capacity, rapid engagement of stretch receptors, alterations in gastric motility, pressure induced alteration in gut hormone levels, and alterations to the flow of food either into or out of the stomach.

With each of the above-described stomach distension devices, safe, effective treatment requires that the device be regularly monitored and adjusted to vary the degree of distension applied to the stomach.

Implants such as those described above include electronics which require a power source that is sufficient for the intended function, such as making adjustments to the gastric coil. Such devices may be internally powered by a battery or capacitor while others may be powered by an externally coupled power source or passive telemetry system. When coupling externally, the efficiencies between the implant and external device diminish substantially as the distance between them increases. There can also be significant power losses through tissue.

Accordingly, there is a need for methods and devices for charging implanted electronics efficiently through tissue by using internal, external and/or non-invasive techniques. It would also be advantageous for a patient to be able to recharge implants without having to travel to a scheduled clinician visit.

### BRIEF DESCRIPTION OF THE DRAWINGS

The invention will be more fully understood from the following detailed description taken in conjunction with the accompanying drawings, in which:

FIG. 1 is a representation of a food intake distension system implanted in a patient to cause a distension in the patient's stomach;

FIG. 2 is representation of a thermoelectric powering device for powering the food intake restriction system of FIG. 1;

FIG. 3 is a representation of one embodiment of a kinetic motion powering device for powering the stomach distension system of FIG. 1;

FIG. 4 is a representation of another embodiment of a kinetic motion powering device for powering the stomach distension system of FIG. 1; and

FIG. 5 is a representation of still another embodiment of a kinetic motion powering device for powering the stomach distension system of FIG. 1.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention provides methods and devices for providing power to an implantable distension system. In one exemplary embodiment, a system for forming a distension in a patient is provided and includes an implantable distension device adapted to cause a distension in the stomach within a patient. For example, the implantable distension device can include a gastric coil and a housing in communication with the gastric coil. The implantable distension device can also include a communicating member that powers the implantable distension device. The system can further include an external apparatus that is operable to communicate with the communicating member by sending power and/or data signals to the communicating member and/or by receiving data signals from the communicating member. The communicating member can also be configured to send data signals to an external device. The external apparatus can optionally include a gauge that is effective to indicate whether the external apparatus is effectively communicating with the communicating member. The system may also be adjustable. Exemplary non-limiting examples of adjustable implantable distension devices (e.g., satiation and satiety inducing gastric implants), optimal design features, as well as methods for installing and removing them are described in commonly owned and pending U.S. patent application Ser. No. ______, filed on even date herewith and entitled “Devices and Methods for Adjusting a Satiation and Satiety-Inducing Implanted Device” [Atty. Docket No. END6514USNP], which is hereby incorporated herein by reference in its entirety.

In one embodiment, the communicating member can be adapted to utilize a temperature differential to power the implantable distension device, the energy transfer apparatus can have a temperature source operable to create a temperature differential across the communicating member to power the implantable distension device. In an exemplary embodiment, the communicating member is a thermogenerator. The temperature source can be, for example, ice, a thermoelectric cooler, a heating source, and a blood vessel. The communicating member can be configured to utilize a temperature differential between the temperature source and an anatomical reference temperature to produce energy to power the implantable distension device. In another embodiment, the gauge can be effective to indicate whether a temperature differential exists between the temperature source and the communicating member effective to power the implantable distension device.

In a preferred embodiment, the communicating member can have a kinetic motion apparatus operable to convert motion into energy to power the implantable distension device. The kinetic motion apparatus can include a housing, a magnet disposed within the housing, and a wire coil disposed around the housing. The wire coil can be in electrical communication with the implantable distension device and the magnet can be configured to move relative to the wire coil to create electrical energy to power the implantable distension device. The kinetic motion apparatus can further include a storage device for storing the electrical energy produced from movement of the magnet. Alternatively, the system may incorporate a piezo-electric device which may convert oscillations into electrical energy. The system may also include an external device that may include a driver adapted to produce corresponding oscillations, vibrations, or other motions in the kinetic motion apparatus effective to power the implantable distension device. Alternatively, an external oscillating electromagnet can induce sympathetic oscillations in the magnet disposed within the housing. In another embodiment, the gauge can be adapted to indicate a charge status of the implantable distension device.

In a further exemplary embodiment, a kinetic motion apparatus can include a counterweight coupled to a drive gear and configured to rotate freely about a pivot point when the kinetic motion apparatus is rotated in response to patient movement. The kinetic motion apparatus can also include an electric generator configured to receive mechanical energy from the drive gear and convert it to electrical energy to power the implantable distension device.

In one embodiment, a kinetic motion apparatus can include a piezoelectric device configured to convert internal muscle and/or organ movement within a patient into electrical energy to power the implantable distension device. The piezoelectric device can also be configured to convert digestive movement of a patient's stomach against the gastric coil into electrical energy to power the implantable distension device.

Methods are also provided for powering an implantable distension device. In one embodiment, a method is provided for powering an implantable distension device and includes placing a temperature source on a tissue surface adjacent to a communicating member disposed within an implantable distension device implanted in a patient. The communicating member utilizes a temperature differential to power the implantable distension device. The communicating member may be placed in the stomach such that it resides in a temperature gradient between the external environment and the body core when an extreme temperature in food is ingested. Alternatively, the thermogenerator can be placed in contact with a large blood vessel since the body uses the blood stream to convey heat to and from the body. Thus, a natural temperature gradient exists in the body with may be used to generate power. The temperature source can be on an external device, and the external device can receive data from the communicating member. The external device can also include a gauge that indicates whether a temperature differential exists between the temperature source and the communicating member effective to power the implantable distension device. The data can include at least one measurement of pressure of fluid within the implantable distension device. In one embodiment, the temperature source can be ice, a thermoelectric cooler, and/or a heating source placed on or near a tissue surface adjacent to the thermogenerator creating a temperature differential with an anatomical reference temperature across the thermogenerator to produce electrical current to power the implantable distension device.

In a preferred embodiment, a method for providing power to an implantable distension device is provided and includes driving a communicating member coupled to an implantable distension device implanted in a patient to power the implantable distension device, where the communicating member includes a kinetic motion apparatus. The kinetic motion apparatus can include a metal coil and a magnet and the metal wire and a magnetic field created by the magnet move relative to one another, thereby generating electrical energy to power the implantable distension device. In an exemplary embodiment, the metal coil and the magnetic field move relative to one another in response to motion by the patient. The kinetic motion apparatus can also be driven by an external oscillating electromagnet that induces sympathetic oscillations in the magnet. Alternatively, the kinetic motion apparatus is driven by a vibration element that causes the metal wire to move through the magnetic field. In another embodiment, the communicating member can be in communication with an external device that receives data from the communicating member and which can include a gauge that indicates a charge status of the implantable distension device. The kinetic motion apparatus can alternatively include a counterweight coupled to a drive gear that rotates freely about a pivot point in response to patient movement. Rotation of the counterweight and drive gear can generate mechanical energy that is converted into electrical energy to power the implantable distension device. In one exemplary embodiment, the kinetic motion apparatus includes a piezoelectric device that converts internal muscle and/or organ movement within a patient into energy to power the implantable distension device. The piezoelectric device can also convert digestive motion of the stomach against the gastric coil into electrical energy to power the implantable distension device. The method can include storing excess energy generated by the kinetic motion apparatus in a storage device.

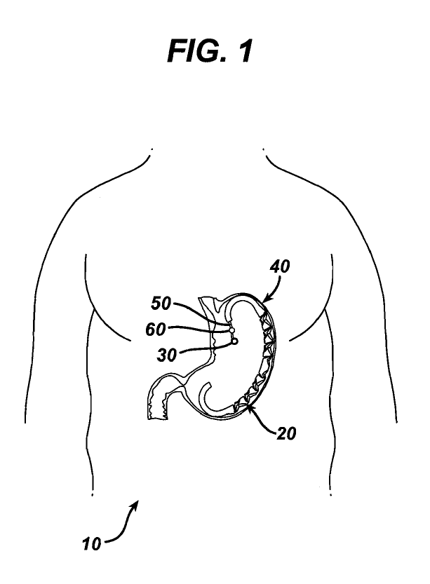

Various powering devices are provided for transferring energy from an external source through tissue to a communicating member implanted in a patient. The energy transferred to the communicating member can be used to provide power to an implantable distension device that is implanted to cause a distension in the stomach within a patient. While the present invention disclosed herein can be used with a variety of implantable distension devices known in the art, FIG. 1 illustrates one exemplary embodiment of a stomach distension system 10. As shown, the system 10 generally includes an adjustable gastric coil 20 that is configured to be positioned in the patient's stomach 40. In addition, the system 10 can include a communicating member capable of providing power to various devices configured to perform any number of tasks within the system 10, as will be described below.

The communicating member can be located anywhere in the system 10. For example, in one embodiment, the communicating member can be disposed within an injection port 30 shown in FIG. 1. The injection port 30 can be in fluid communication with the gastric coil for allowing fluid to be introduced into and removed from the coil to alter the amount of distension provided by the coil. Alternatively, or in addition, the communicating member can be disposed within a housing 60 that can house various components. In the illustrated embodiment, the system 10 includes both an injection port 30 and a housing 60. Both the injection port 30 and the housing 60 are coupled to the adjustable gastric coil 20, e.g., via a catheter 50. A person skilled in the art will appreciate that the system need not include an injection port and/or housing, and that the communicating member can be positioned anywhere along the system 10.

In an exemplary embodiment, the communicating member can convert energy received from an external source to provide power to devices within the system 10 that measure and/or monitor various conditions of the system 10, that make adjustments to the gastric coil 20 and/or other aspects of the system 10, and/or that measure/monitor various physiological parameters. Such devices can include, for example, sensors, pumps, coils and/or any other monitoring and/or adjustment devices having circuitry which requires electrical power. The communicating member can be configured to repeatedly receive energy from an external source, convert the energy to electrical power, and store the power in a capacitor, battery or other power storage device known in the art for later use by the device(s) within the system 10. Alternatively, the communicating member can be configured to transfer the converted power directly to the device(s) as needed. In addition, the communicating member can be configured to transmit and receive data to and from an external source. For example, the communicating member can receive command signals from an external source related to powering the system 10. The communicating member can also transmit various anatomical measurements taken within a patient's body to an external device or reader, as well as to transmit information regarding the charge status of the system 10.

The communicating member can take any form known in the art, and various embodiments of the communicating member are provided in detail below. In certain exemplary embodiments, the communicating member can take the form of a sensor capable of receiving energy from an external source for measuring and monitoring various parameters of the system 10; an antenna such as a dipole antenna, a monopole antenna with appropriate counterpoise, or an inductive coil capable of receiving energy through tissue; and/or any other devices known in the art which are capable of aiding in the powering, measuring, monitoring, and/or adjusting of the system 10 and/or other physiological parameters associated with the system 10.

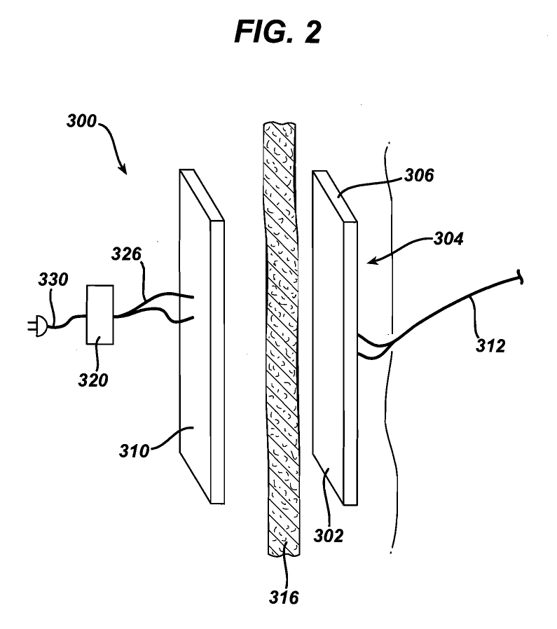

In one exemplary embodiment shown in FIG. 2, the communicating member can be in the form of a thermoelectric generator 306, such as a Peltier device, configured to use a temperature differential to generate electricity. The external device can include a thermoelectric powering device 300 adapted to power an implantable distension device implanted within a patient. In an exemplary embodiment, the generator 306 can be implanted under a patient's skin and a temperature differential can be created across the generator 306 by providing an external temperature source which is different than body temperature. As shown, the generator 306 includes a first side 302, which faces outward from the patient's body and is positioned in the stomach. The generator 306 also includes a second side 304 which faces towards in the opposite direction. The generator 306 includes electrical leads 312 which can be connected to a storage device, such as a capacitor or battery, or directly to the devices within the implantable distension device. A means for monitoring the charge level of the storage device may also be included.

As shown in FIG. 2, the thermoelectric powering device includes a temperature source 310. A person skilled in the art will appreciate that the temperature source 310 can be any device or element which is capable of producing a temperature that is different than the temperature associated with the second side 304 of the generator 306. For example, if the temperature of the second side 304 of the generator 306 is at an anatomical reference temperature such as a human body temperature, then the temperature source 310 can be a piece of ice which is at a temperature cooler than the anatomical reference temperature. Alternatively, the first side 302 of the generator 306 can be placed in contact with a large blood vessel within the body, since the body uses the blood stream to convey heat to and from the body. A natural temperature gradient exists in the body between the blood vessel and the body, and therefore between the first side 302 and the second side 304, which can be used to generate power.

In an exemplary embodiment, in use, when a patient or physician places the temperature source 310, e.g. ice, against a tissue surface 316, in proximity to the first side 302 of the implanted generator 306, a temperature differential is created across the generator 306, thereby causing it to generate electricity. A patient and/or physician can place the temperature source 310 against an area of the patient's skin that covers the first side 302 of the implanted generator 306. The temperature source 310 will change the temperature of the first side 302 of the generator so that there is a difference in temperature between the first side 302 and the second side 304 effective to generate electricity. In another example, the temperature source 310 can be a second Peltier device used as a thermoelectric cooler so that one side of the device is much cooler than the temperature of the second side 304 of the implanted generator 306. The thermoelectric cooler can then be placed adjacent to the tissue surface 316 in proximity to the first side 302 of the implanted generator 306, thereby creating a temperature differential across the generator 306 to produce electricity. Alternatively, the temperature source 310 can be eddy-current heating of a conductive component connected to or within the implantable restrictive device. The eddy current may be generated by an inductive coupled external alternating power source. Heating may be controlled for example by the mass of the conductive component, the size and shape of the component, magnetic permeability of the conductive component, resistivity of the conductive component, external power coupling frequency or the external power output level, etc. In one exemplary embodiment, the heat source could be a heating pad placed on or near the tissue surface. The electricity which is generated can then be used by devices within the implantable distension device as needed.

The temperature source 310 can alternatively be connected to or disposed within an external device 320. The external device 320 can include a gauge that indicates whether a temperature differential exists between the temperature source 310 and the generator 306 that is effective to charge and/or power the implantable distension device. The indication given by the gauge can take the form of any notification means known in the art, including a light, such as an LED, an audible noise, and/or a vibration. If the temperature source 310 is ice or another temperature element which doesn't require electrical power, an external device 320 may not be required for the purpose of providing power. If the temperature source 310 is a thermoelectric cooler or other electrically powered temperature source as illustrated in FIG. 2, then the external device 320 can provide power to the temperature source 310 via electrical leads 326. The external source 320 can contain batteries or other power source, or can be connected to a wall power source via cable 330.

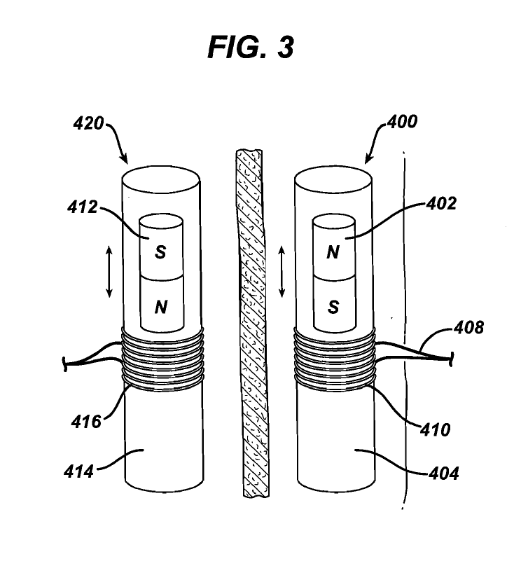

FIG. 3 shows another embodiment of a communicating member in the form of a kinetic motion apparatus 400 adapted to provide power to the implantable distension device. In one exemplary embodiment, as shown, the kinetic motion apparatus 400 includes a housing having a magnet 402 disposed therein. The housing can be of any shape and made of any material known in the art, but in the illustrated embodiment, the housing is in the form of a glass tube or cylinder 404 having a metal or copper wire 410 wrapped tightly in a coil around an exterior surface of the cylinder 404. In this configuration, the kinetic motion apparatus 400 can generate electricity in the copper wire 406 by movement of the magnet 402 contained within the cylinder 404. Movement of the magnet 402 within the cylinder 404 will effectively cause the copper wire 410 to be moved through a magnetic field, thereby causing electricity to be generated, as will be appreciated by those skilled in the art. Electrical leads 408 coupled to the copper wire 410 are provided to carry the electricity generated by the kinetic motion apparatus 406 to a storage device or directly to devices within the implantable distension device as needed. A means for monitoring the charge level of the storage device may also be included.

While many configurations are possible, in one exemplary embodiment, the kinetic motion apparatus 400 can be implanted within a patient's body such that physical movement of the body is effective to move the magnet 402 within the cylinder 404. For example, a patient can perform any movement, such as walking, running, jumping, shaking, etc., and this will cause the magnet 402 to move laterally, rotationally, or any combination thereof, within the cylinder 404 to generate electricity within the copper wire 406. In another example, the kinetic motion apparatus 400 may be implanted within a patient's body such that more subtle, but predictable physical movements within the body are effective in moving the magnet 402 within the cylinder 404. Examples of internal movements within the patient that may be harnessed include, but are not limited to, motions related to respiration (e.g., motions of the diaphragm), digestion (e.g., peristaltic waves through any portion of the gastrointestinal tract), and/or oscillatory motions within the circulatory system (e.g., pulsatile flow in the arterial system, motion of the heart, etc.).

Alternatively, or in addition, the kinetic motion apparatus 400 can include an external driver. In the embodiment shown in FIG. 3, the external driver is composed of the same elements as the kinetic motion apparatus 400, namely, a housing 414, a magnet 412, and a copper wire 416 to form an external electromagnet 420. The external electromagnet 420 can be manually driven by supplying the copper wire 416 with electricity to cause the magnet 412 to oscillate. As the magnet 412 oscillates, sympathetic oscillations are induced in the magnet 402 disposed within the kinetic motion apparatus 400, thereby causing electricity to be generated to supply power to the implantable distension device. A person skilled in the art will appreciate that any driver or vibration element, internal or external, which is effective to produce oscillations, vibrations, or other motions in the magnet 402 within the kinetic motion apparatus 400, can be used to generate power. One additional alternative may include the conversion of oscillatory gradients in pressure created by natural and regularly occurring events such as respiration into fluid flows that induce oscillatory translational and/or rotational motions of the magnet 402. Moreover, the kinetic motion apparatus 400 can have a variety of other configurations in which energy is generated from motion or pressure gradients caused by these motions.

Although not shown in FIG. 3, an external device can also be provided to be in communication with the external driver and it can provide power to the external driver taken from a battery or other power source. The external driver can also include a gauge that indicates a charge status of the communicating member and/or whether there is proper alignment between an external driver and the kinetic motion apparatus 400. For example, the gauge can indicate whether circuitry and/or devices within the implantable distension device need to be charged by the kinetic motion apparatus 400, or whether they are fully charged. Alternatively or in addition, the gauge can indicate proper alignment of an external driver that is attempting to generate sympathetic oscillations within the kinetic motion apparatus 400. The indication given by the gauge can take the form of any notification means known in the art, including a light, such as an LED, an audible noise, and/or a vibration.

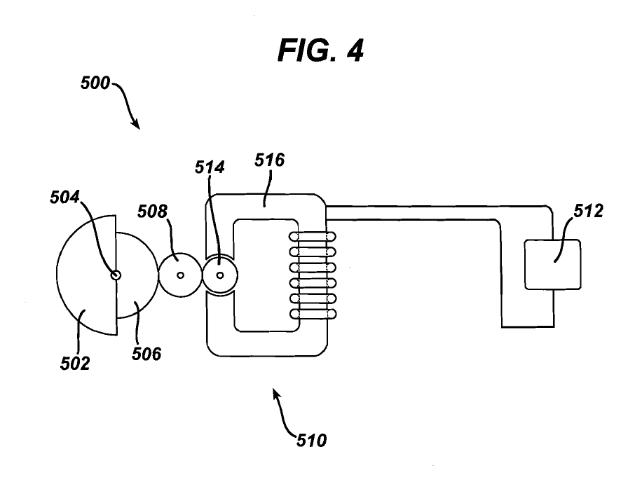

In another exemplary embodiment, a kinetic motion apparatus is provided that is operable to convert motion into energy to power the implantable distension device. In one embodiment shown in FIG. 4, a kinetic motion apparatus 500 is provided and can include a counterweight 502 coupled to a shaft 504 such that the counterweight 502 can freely pivot about the shaft 504 in response to motion and movement of the patient. The counterweight 502 and the shaft 504 can be formed from any biocompatible material known in the art, including stainless steel, titanium, cobalt chrome, and any number of polymer plastics. A drive gear 506 can be nested within a hollow portion of the counterweight 502, and in one embodiment, it can be directly coupled to the counterweight 502 such the drive gear 506 moves in response to movement of the counterweight 502. The drive gear 506 can also be coupled to a drive train of an electric generator 510. As the drive gear 506 moves in response to the counterweight 502, it rotates a pinion gear 508 which in turn rotates the rotor 514 to a high velocity. This rotation then induces electric current through the stator 516 thereby charging the capacitor 512. The electric generator 510 thus converts mechanical energy from movement of the counterweight 503 into electrical energy.

The electrical energy produced by the generator 510 can be used to directly power the implantable distension device or it can be stored within an accumulation element 512 for later use. In an exemplary embodiment, the accumulation element 512 can be a capacitor that contains lithium ion which provides an efficient conducting surface that may store energy longer than those capacitors typically made from other substrates. In another embodiment, the accumulation element 512 can be a high density ultracapacitor. A person skilled in the art will appreciate that any combination of gearing can be used to couple a patient's movement to the generator and any type of accumulation element 512 can be used to store charge.

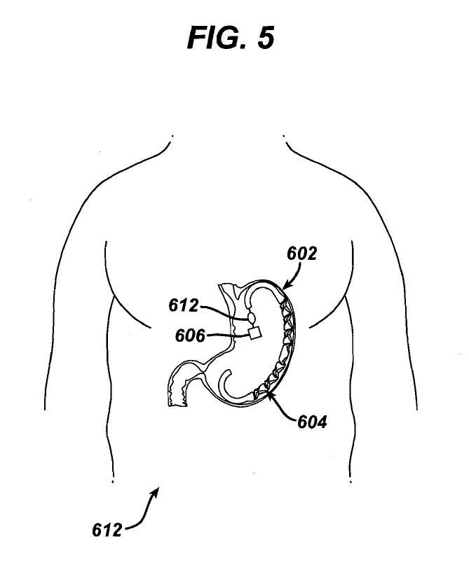

In another embodiment shown in FIG. 5, a kinetic motion apparatus 600 is provided such that motion of a stomach 602 pushing against fluid in the gastric coil 604 is converted into energy to supply power to a rechargeable battery or an accumulation element 606 that stores charge. As food passes through the coil 604, pressure will increase and decrease in the gastric coil 604. This vibration energy can be harvested by a variety of different methods known in the art such as electromagnetic, electrostatic, or piezoelectric conversion. In piezoelectric (piezo) methods, a bimorph based on piezoelectric materials vibrates, creating a charge that generates a voltage with amplitude proportional to the size and shape of the piezoelectric material, periodicity, and amount of force. Thus, the kinetic motion apparatus 600 can include a piezoelectric transducer element 612 attached to the gastric coil 604 that can produce power proportional to the displacement and periodicity of coil movement. This energy can then be stored in the accumulation element 606 until needed by the implantable distension device. A person skilled in the art will appreciate that similar use can be made of electro-active polymer elements attached to the gastric coil.

An internal battery can be created with an anode and cathode placed within the gastric lumen. The gastric acids will help to create an electrical potential between two dissimilar metals (gold=0.0 and magnesium=1.75 on the anodic index). The galvanic response will corrode the magnesium (which is biocompatible) in favor of the gold or gold plated element. Alternatively silver, nickel, or titanium may replace the gold element.

The internal devices disclosed herein are designed to be single use devices. The external devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application. The implantable devices disclosed herein are designed for single patient use.

Any patent, publication, application or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A device, including an implant for placement within a hollow body organ, said device comprising:
a. a distension device having an undeployed shape for delivery within a hollow body and one or more deployed shapes for implantation therein;
b. said distension device having sufficient rigidity in its deployed shape to exert an outward force against an interior of the hollow body so as to bring together two substantially opposing surfaces of said hollow body; and
c. a powered means for changing the deployed shape of said member while implanted within said hollow body including an apparatus operable to convert energy produced by the patient into energy to power the implantable device.

2. The system of claim 1 wherein said apparatus converts kinetic energy into electrical energy.

3. The device of claim 1 wherein said apparatus converts thermal energy into electrical energy.

4. The device of claim 1 wherein the implantable device is adapted to cause a distension in the stomach of a patient

5. The device of claim 1, wherein the kinetic motion apparatus includes a housing and a magnet disposed within the housing.

6. The device of claim 5, wherein the kinetic motion apparatus further includes a wire coil disposed around the housing.

7. The device of claim 6, wherein the wire coil is in electrical communication with the implantable distension device.

8. The device of claim 6, wherein the wire coil includes a ferrite core to enhance or augment the coupling.

9. The device of claim 5, wherein the magnet is configured to move relative to the wire coil to create electrical energy to power the implantable distension device.

10. The device of claim 9, wherein the kinetic motion apparatus further includes a storage device for storing the electrical energy produced from movement of the magnet.

11. The device of claim 10, wherein the kinetic motion apparatus includes a counterweight coupled to a drive gear and configured to rotate freely about a pivot point when the kinetic motion apparatus is rotated in response to a patient's movement.

12. The device of claim 11, wherein the kinetic motion apparatus further includes an electric generator configured to receive mechanical energy from the drive gear and convert it to electrical energy to power the implantable distension device.

13. The device of claim 10, wherein the kinetic motion apparatus includes a piezoelectric device configured to convert internal muscle and/or organ movement within a patient into electrical energy to power the implantable distension device.

14. The device of claim 1, further comprising an external device adapted to send or receive data from the implantable distension device.

15. The device of claim 1, wherein the communicating member is configured to receive and transmit data.

16. The device of claim 14, wherein the external device includes a gauge effective to indicate a charge status of the implantable distension device.

17. The device of claim 10, further comprising a driver adapted to produce motion of at least one portion of the kinetic motion apparatus effective to power the implantable distension device.

18. The device of claim 5, further comprising an external oscillating electromagnet effective to induce sympathetic oscillations in the magnet disposed within the housing.

19. The device of claim 1, wherein the implantable distension device comprises a gastric coil and a housing in communication with the gastric coil.

20. The device of claim 19, wherein the kinetic motion apparatus includes a piezoelectric device configured to convert digestive movement of a patient's stomach against the gastric coil into electrical energy to power the implantable distension device.
